(19) United States
(12) Patent Application Publication
(10) Pub. No.: US 2020/0289412 A1
(43) Pub. Date: Sep. 17, 2020

(54) BIOENHANCED SPIRULINA LOZENGE FORMULATION

(71) Applicant: WELLISEN INC., Reston, VA (US)

(72) Inventors: Jayant DESHPANDE, Edison, NJ (US); Yuvraj PURI, Great Falls, VA (US)

(21) Appl. No.: 16/815,724

(22) Filed: Mar. 11, 2020

Related U.S. Application Data
(60) Provisional application No. 62/816,338, filed on Mar. 11, 2019.

(51) Int. Cl. A61K 9/00; A61K 35/748; A61K 36/37; A61K 36/48; A61K 36/886; A61K 47/36; A61K 47/10; A61K 47/12; A61K 47/55; A61K 9/20

(52) U.S. Cl. CPC A61K 9/0056; A61K 35/748; A61K 36/37; A61K 36/48; A61K 36/886; A61K 9/20; A61K 47/10; A61K 47/12; A61K 47/551; A61K 9/006; A61K 47/36

(57) ABSTRACT

An oral dosage form of Spirulina aqueous extract is described here for delivering medications to alleviate symptoms of influenza virus. Most potent antiviral principles of spirulina being polysaccharides with molecular weight ranging from 100-300 kDa, there is need to provide permeation enhancers that help these polysaccharides in crossing the mucus membrane of oropharynx. Apart from enhancing the permeation, the formulation provides sustained effect to ameliorate symptoms of cold, cough, headache, fever related to influenza viral infection.

### FIELD OF INVENTION

Present invention describes a formulation of spirulina extract for the amelioration of discomfort and intensity of influenza infection. Particularly, this invention describes an aqueous extract of spirulina for the antiviral effect against influenza. More particularly, an aqueous extract of spirulina which is rich in high molecular weight polysaccharide is formulated to treat influenza virus. Instant invention describes a lozenge formulation of polysaccharide, which is most desired route of administration for anti influenza medication. A lozenge formulation targets the actual site of influenza infection, namely oropharyngial region. Formulation in the form of lozenge delivers anti-viral medication directly to the body tissues which are heavily infected with influenza virus. Lozenge dissolves slowly and steadily, releasing the bioactives in a sustained, controlled manner, maximizing the benefits of the treatment. Since the efficacy of antiviral medication in the form of lozenge depends on the ability of the bioactives in permeating through the mucus membrane of the oropharyngial region, the instant invention provides a novel lozenge formulation that allows quicker, more efficient permeation of bioactives whose molecular weights are in the range of 100,000 to 300,000 kDa.

### BACKGROUND

Spirulina represents a biomass of cyanobacteria (blue-green algae) that can be consumed by humans and other animals. The two species are Arthrospira platensis and A. maxima.

The species A. maxima and A. plaetensis were once classified in the genus Spirulina. The common name, spirulina, refers to the dried biomass of A. platensis, which belongs to photosynthetic bacteria that cover the groups Cyanobacteria and Prochlorophyta. Scientifically, a distinction exists between spirulina and the genus Arthrospira. Species of Arthrospira have been isolated from alkaline brackish and saline waters in tropical and subtropical regions. Among the various species included in the genus Arthrospira, A. platensis is the most widely distributed and is mainly found in Africa, but also in Asia. A. maxima is believed to be found in California and Mexico. The term spirulina remains in use for historical reasons.

Arthrospira species are free-floating, filamentous cyanobacteria characterized by cylindrical, multicellular trichomes in an open left-handed helix. They occur naturally in tropical and subtropical lakes with high pH and high concentrations of carbonate and bicarbonate. A. platensis occurs in Africa, Asia, and South America, whereas A. maxima is confined to Central America. Most cultivated spirulina is produced in open-channel raceway ponds, with paddle wheels used to agitate the water.

Provided in its typical supplement form as a dried powder, a 100-g amount of spirulina supplies 290 Calories and is a rich source (20% or more of the Daily Value, DV) of numerous essential nutrients, particularly protein, B vitamins (thiamin and riboflavin, 207% and 306% DV, respectively), and dietary minerals, such as iron (219% DV) and manganese (90% DV). The lipid content of spirulina is 8% by weight providing the fatty acids, gamma-linolenic acid, alpha-linolenic acid, linoleic acid, stearidonic acid,] eicosapentaenoic acid (EPA), docosahexaenoic acid (DHA), and arachidonic acid. In contrast to those 2003 estimates (of DHA and EPA each at 2 to 3% of total fatty acids), 2015 research indicated that spirulina products “contained no detectable omega-3 fatty acids” (less than 0.1%, including DHA and EPA). An in vitro study reported that different strains of microalgae produced DHA and EPA in substantial amounts.

Allergy, Rhinitis, and Immunomodulation: It has been well documented that Spirulina exhibits anti-inflammatory properties by inhibiting the release of histamine from mast cells.

In a recent randomized, double-blind placebo-controlled trial, individuals with allergic rhinitis were fed daily, either with placebo or Spirulina for 12 weeks. Peripheral blood mononuclear cells were isolated before and after the Spirulina feeding and levels of cytokines (interleukin-4 (IL-4), interferon-γ (IFN-γ) and interleukin-2), which are important in regulating immunoglobulin (Ig)E-mediated allergy, were measured. The study showed that high dose of Spirulina significantly reduced IL-4 levels by 32%, demonstrating the protective effects of this microalga toward allergic rhinitis.

Ishii et al. studied the influence of Spirulina on IgA levels in human saliva and demonstrated that it enhances IgA production, suggesting a pivotal role of microalga in mucosal immunity.

A Japanese team identified the molecular mechanism of the human immune capacity of Spirulina by analysing blood cells of volunteers with pre- and post-oral administration of hot water extract of Spirulina platensis. IFN-γ production and Natural Killer (NK) cell damage were increased after administration of the microalga extracts to male volunteers.

In a recent double-blind, placebo-controlled study from Turkey evaluating the effectiveness and tolerability of Spirulina for treating patients with allergic rhinitis, Spirulina consumption significantly improved the symptoms and physical findings compared with placebo, including nasal discharge, sneezing, nasal congestion and itching.

It is well understood that deficiency of nutrients is responsible for changes in immunity, which manifests as changes in production of T-cells, secretory IgA antibody response, cytokines and NK-cell activity. The above studies suggest that Spirulina may modulate the immune system by its role in covering nutritional deficiencies.

### Antiviral Applications: In Vitro Studies

There are no in vivo studies providing strong evidence supporting the possible antiviral properties of Spirulina. The active component of the water extract of S. platensis is a sulfated polysaccharide, calcium spirulan (Ca-Sp). According to Hayashi et al, Ca-Sp inhibits the in vitro replication of several enveloped viruses including Herpes simplex type I, human cytomegalovirus, measles and mumps virus, influenza A virus and human immunodeficiency virus-1 virus (HIV-1).

Another more recent study showed in vitro that an aqueous extract of S. platensis inhibited HIV-1 replication in human T-cells, peripheral blood mononuclear cells and Langerhan cells. The advantage of using herbs and algal products with proven antiviral properties in fighting certain viruses is that they can be used-through immunomodulation-even when the infection is established.

According to Yi-Hsiang Chen (Yi-HsiangChen 2016 (Scientific Reports|6:24253|DOI: 10.1038/srep24253) Spirulina cold water extract, which revealed the contents to be 39.33±5.6% of protein, 11.79±5.7% of polysaccharides, 19.29±2.7% of nucleic acids, 5±1% of water, 1.2±0.3% of ash, and 23.39% of other or unknown components, has demonstrated significant antiviral effect. C-phycocyanin makes up about 50% of the protein fraction would be a major component of the Spirulina cold water extract, while allophycocyanin takes up about 10%. The results of the neutralization tests suggest that the active compound(s) responsible for anti-influenza activity are likely to be high molecular weight (>100 kDa), heat-susceptible, and negatively charged polysaccharide(s)

Spirulina cold water extract has low cellular toxicity, and is well-tolerated in animal models at one dose as high as 5,000 mg/kg, or 3,000 mg/kg/day for 14 successive days. Anti-flu efficacy studies revealed that the Spirulina extract inhibited viral plaque formation in a broad range of influenza viruses, including oseltamivir-resistant strains. Spirulina extract was found to act at an early stage of infection to reduce virus yields in cells and improve survival in influenza-infected mice, with inhibition of influenza hemagglutination identified as one of the mechanisms involved. Together, these results suggest that the cold water extract of Spirulina might serve as a safe and effective therapeutic agent to manage influenza outbreaks, and further clinical investigation may be warranted.

U.S. Pat. No. 5,585,365 describes a method for prophylactic or therapeutic treatment of viral diseases. The spirulina extract comprises rhamnose, glucose, fructose, ribose, galactose, xylose, mannose, glucuronic acid and galacturonic acid; it exhibits an absorption at 480 nm in phenolsulfuric acid reaction; and it has a molecular weight of 250,000 to 300,0000 as determined by gel filtration method.

Binyui Wang (Interational Journal of Molecular Medicine 42: 1273-1282, 2018) describes Spirulina polysaccharide (PSP), a type of water-soluble, physiologically active polysaccharide extracted from spirulina, that has a large and complex molecular structure, which is mainly composed of glycosidic bonds. PSP is reported to have an effect on inhibiting tumor cell growth through inhibiting the synthesis of nucleic acid and proteins in cancer cells, but not directly killing cancer cells. In addition, the inhibitory effect of PSP on cancer cells has been reported to be time-dependent. It is well known that free radicals can oxidize biomolecules and are important in several degenerative and pathological processes. As an antioxidant, PSP can maintain cellular health and inhibit senescence in the body by removing excess free radicals and preventing the oxidation of cellular oxidative substrates. PSP can enhance the non-specific cellular immune function in the body, and improve the ability to resist the invasion of viruses.

### SUMMARY

While there are large number of publications describing significant anti influenza activity of spirulina aqueous extract that contains polysaccharides in in-vitro (cell-line) studies, to the best of our knowledge, there are no publications that demonstrate in-vivo efficacy. Many efforts in this direction may have not been successful for the reason that the supplement/drug administered by oral route is not reaching the tissues afflicted by the virus in adequate quantities. Presumably, polysaccharides are digested in gastrointestinal tract and their antiviral activity may have been diminished.

Major route of entry for influenza virus is through nasopharynx. From there, it may travel to other organs such as trachea and lungs. Other organs that may be affected due to the virus are sinuses, brain, eyes and joints. Any antiviral therapy would be effective if the active principles reach these target organs.

There are references available in the literature that describe lozenge application of spirulina spray dried powder, which however are not useful in treating the influenza virus for two reasons, 1) these products are not rich in high molecular weight polysaccharides, which have potent anti-viral activity and 2) the conventional lozenge formulation will not allow high molecular weight active principles to cross the mucosal barrier of nasopharynx and reach tissues afflicted by the influenza virus.

The present inventors have surprisingly found that certain food and pharmaceutical grade bioenhancers have the ability to enhance the permeability of high molecular weight polysaccharides and proteins through the mucus membrane of the oropharynx.

### DETAILED DESCRIPTION

Influenza virus affects numerous tissues in the body which include oropharynx, sinus, brain, eyes, trachea and lungs. Delivering an antiviral supplement/drug to these organs has always been a challenge. Conventional oral route has several limitations such as acid degradation, poor bioavailability, extensive degradation in the first pass metabolism, inactivation during metabolism, excessive protein binding, quick elimination, to name a few.

Due these disadvantages, topical delivery has gained importance. The main advantage of topical delivery is that it reaches the affected parts, namely oropharynx, nasopharynx, fairly quickly. Another important advantage being delivering the bioactives bypassing the metabolism. Since most bioactives get metabolized extensively and the resulting metabolites being less active, topical delivery which avoids interaction with microsomal enzymes, provides significant advantage.

The bioenhanced lozenge containing spirulina extract described herein is made by formulating spirulina water soluble extract with food or pharmaceutical grade excipients and with at least two additives that enhance absorption of high molecular weight polysaccharides.

In addition to enhancing the permeability of polysaccharides, added bioenhancers may also increase absorption on non-carbohydrate constituents of spirulina such as proteins and minerals.

Unless defined otherwise, all technical and scientific terms used herein have the same meaning as is commonly understood by one of skill in the art. All patents and publications referred to herein are incorporated by reference.

As used in the specification and claims, the singular form “a,” “an,” and “the” include plural references unless the context clearly dictates otherwise.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

The terms “treatment,” “treating,” “palliating,” and “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal so that both agents and/or their metabolites are present in the animal at the same time. Co-administration includes simultaneous administration in separate compositions, administration at different times in separate compositions, or administration in a composition in which both agents are present.

A “pharmaceutically acceptable salt” means a salt composition that is generally considered to have the desired pharmacological activity, is considered to be safe, non toxic and is acceptable for veterinary and human pharmaceutical applications. Pharmaceutically acceptable salts may be derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions described herein is contemplated. Supplementary active ingredients can also be incorporated into the compositions.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. Although antagonists herein generally interact specifically with (e.g. specifically bind to) the target, compounds that inhibit a biological activity of the target protein by interacting with other members of the signal transduction pathway of which the target protein is a member are also specifically included within the definition of “antagonist.”

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. Agonists herein generally interact specifically with (e.g. specifically bind to) the target, compounds that initiate or enhance a biological activity of the target polypeptide by interacting with other members of the signal transduction pathway of which the target polypeptide is a member are also specifically included within the definition of “agonist.”

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize the limits to the structural nature of the agents described herein.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

The term “selective inhibition” or “selectively inhibit” as applied to a biologically active agent refers to the agent's ability to selectively reduce the target signaling activity as compared to off-target signaling activity, via direct or interact interaction with the target.

“Subject” refers to an animal, such as a mammal, for example a human. The methods described herein can be useful in both human therapeutics and veterinary applications. In some embodiments, the patient is a mammal, and in some embodiments, the patient is human.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

The term “spirulina extract” as used herein means an aqueous extract of spirulina, which is enriched with respect to high molecular weight polysaccharides as compared to prior art spirulina powder products. For example, the USFDA lists a typical spirulina powder product as containing, inter alia, 50 g carbohydrate. Accordingly, a spirulina extract as described herein will contain greater than 50 g carbohydrate. In addition to polysaccharides, the extract may also contain protiens such as phycocyanin, vitamins and minerals.

The term “polysaccharide” as used herein means high molecular weight carbohydrates, with molecular weight ranging from 100 k to 300 kDa. However, with actions of bioenhancers, these polysacchrides may get hydrolysed in the mouth resulting in the formation of smaller fragments, some of which may retain antiviral property against influenza virus.

The term ‘influenza virus’ used here includes Type A, B, C and D.

The term “enzyme inhibitor” as used herein refers to any chemical compound which reduces or inhibits the enzymatic degradation of polysaccharides. Examples of enzyme inhibitors that are useful in the products described herein include, but are not limited to, amylase inhibitors, alkaloids, glycosides, flavonoids, carotenoids, polysaccharides, hypoglycans, peptidoglycans, guainidine, steroids, glycopeptides, and terpenoids. Particularly suitable amylase inhibitors for use in the products described herein include, but are not limited to, Salacia extract, white kidney bean extract, alo vera gel, and licorice extract.

The compositions described herein may also comprise a first bioenhancer which increases the absorption of non-carbohydrate consitutents of Spirulina, such as proteins and minerals. These first bioenhancers also increase the trans-cellular permeation of active moieties, such as polysaccharides from Spirulina with a molecular weight of 100-300 kDa. Examples of suitable first bioenhancers include, but are not limited to, Polysorbate 80, Polysorbate 20, glycerol, propylene glycol, polyethylene glycol, polyvinyl pyrrolidone, chitosan hydrochloride, licorice axtract, caprylic acid, sodium caprate, capric acid, and sodium caprate.

The compositions described herein may also comprise a second bioenhancer which reduces the molecular size of polysachharides in the composition, without the loss of antiviral effect. Examples of suitable second bioenhancers include, but are not limited to, alpha amylase, beta amylase, alpho glucosidase, and beta glucosidase.

In yet another embodiment, the permeation enhancer may increase paracellular transport of the active moieties.

Also described herein is a solid pharmaceutical composition for oral administration containing an effective amount of a spirulina extract as described herein. In some embodiments, the pharmaceutical composition may be a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

Also described herein are anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. Pharmaceutical compositions and dosage forms which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, microcrystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions described herein to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teradecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, suitable lipophilic surfactants include, but are not limited to, glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the active ingredients and to minimize precipitation of the compound described herein. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, E lcaprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, E lcaprolactone and isomers thereof, E-valerolactone and isomers thereof, E-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Suitable solubilizers include, but are not limited to, sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Examples may include, but are not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

In a particular embodiment, the compositions described herein are formulated into lozenges. A suitable lozenge formulation may include polysaccharides that are of high molecular weight (>100 Kda) or may also include high molecular weight proteins/peptides.

While embodiments described above may in general describe lozenge as a delivery form, the scope of this invention includes other delivery form applicable for oral cavity, such as mouthwash, gummies, chews, throat paints, sublingual tablets and mucoadhesive delivery forms.

While the compositions and methods herein have been described in terms of specific illustrative embodiments, any modifications and equivalents that would be apparent to those skilled in the art are intended to be included within the scope of the methods and compositions herein. The details of the methods and compositions herein, its objects, and advantages are explained hereunder in greater detail in relation to non-limiting exemplary illustrations.

What is claimed is:

1. An oral dosage form composition comprising an aqueous extract of spirulina and at least two bioenhancers, wherein the spirulina extract is enriched in high molecular weight polysaccharides.

2. The composition of claim 1, wherein at least one bioenhancer is an enzyme inhibitor that reduces the enzymatic degradation of polysaccharides, the enzyme inhibitor selected from the group consisting of amylase inhibitors, alkaloids, glycosides, flavonoids, carotenoids, polysaccharides, hypoglycans, peptidoglycans, guainidine, steroids, glycopeptides, and terpenoids.

3. The composition of claim 2, wherein the enzyme inhibitor is an amylase inhibitor selected from the group consisting of Salacia extract, white kidney bean extract, alo vera gel, and licorice extract.

4. The composition of claim 1, wherein at least one bioenhancer is a compound which increases absorption of non-carbohydrate constituents of spirulina such as proteins and minerals.

5. The composition of claim 4, wherein the bioenhancer is selected from the group consisting of Polysorbate 80, Polysorbate 20, glycerol, propylene glycol, polyethylene glycol, polyvinyl pyrrolidone, chitosan hydrochloride, licorice axtract, caprylic acid, sodium caprate, capric acid, and sodium caprate.

5. The composition of claim 1, wherein the spirulina extract further comprises phycocyanin, vitamins, and/or minerals.

6. The composition of claim 1, wherein the high molecular weight polysaccharides are carbohydrates with a molecular weight of from about 100 kDa to about 300 kDa.

7. The composition of claim 1, wherein at least one bioenhancer is enzyme that reduces the molecular size of the polysaccharide without the loss of antiviral effect.

8. The composition of claim 1, further comprising a permeation enhancer which increases the trans-cellular permeation of active moieties.

9. The composition of claim 8, wherein the active moieties are carbohydrates with a molecular weight of from about 100 kDa to about 300 kDa.

10. The composition of claim 1, wherein the oral dosage form is selected from the group consisting of lozenges, mouthwash, gummies, chews, throat paints, sublingual tablets and mucoadhesive delivery forms.

11. The composition of claim 1, further comprising food or pharmaceutical grade excipients.

12. A method of treating influenza in a patient suffering from influenza, comprising administering a composition of claim 1 to the patient.

13. The method of claim 12, wherein the patient is infected with influenza virus Type A, B, C and/or D.
